(19) United States
(12) Patent Application Publication
(10) Pub. No.: US 2008/0299212 A1
(43) Pub. Date: Dec. 4, 2008

(54) Pharmaceutical Composition for Treating Avellino Cornea Dystrophy Comprising Blood Plasma or Serum

(75) Inventors: Eung Kweon Kim, Seoul (KR); Nae-Choon Yoo, Seoul (KR); Won-Min Yoo, Seoul (KR)

Correspondence Address: INTELLECTUAL PROPERTY / TECHNOLOGY LAW, PO BOX 14329, RESEARCH TRIANGLE PARK, NC, 27709, US

(73) Assignee: MEDIGENES CO., LTD, Seoul (KR)

(21) Appl. No.: 11/816,999

(22) PCT Filed: Feb. 24, 2006
(86) PCT No.: PCT/KR06/00648

§ 371 Date: Aug. 13, 2008

(30) Foreign Application Priority Data
Feb. 25, 2005 (KR) 10-2005-0015612
Feb. 25, 2005 (KR) 10-2005-0015615
Feb. 25, 2005 (KR) 10-2005-0015616
Feb. 25, 2005 (KR) 10-2005-0015620

(51) Int. Cl. A61K 35/16

(52) U.S. Cl. 424/530; 424/531

(57) ABSTRACT

The present invention relates to a pharmaceutical agent for treating Avellino corneal dystrophy, and more particularly, to a pharmaceutical composition for treating Avellino corneal dystrophy comprising pharmaceutically effective amount of blood plasma or serum as an active ingredient. The pharmaceutical composition of the present invention has an effect of improving symptoms by dissolving away hyaline granules in the cornea of a patient with severe Avellino corneal dystrophy due to LASIK surgery.

### TECHNICAL FIELD

The present invention relates to a medicine for treating Avellino corneal dystrophy (ACD), and more particularly, to a pharmaceutical composition for treating Avellino corneal dystrophy comprising pharmaceutically effective amount of blood plasma or serum as an effective ingredient.

### BACKGROUND ART

Avellino corneal dystrophy is a hereditary disease which white granules, hyaline in the cornea of the eye forms milky deposits, so that the cornea becomes blurry to cause bad visual acuity and thus leading to the loss of eyesight (FIG. 1). This disease is generated by point mutation in which codon CGC (arginine) corresponding to 124^{th} amino acid in βIG-H3 gene is replaced by CAC (histidine) (Munier, F. L. et al., Nat. Genet., 15:247, 1997). All people with this abnormal gene show symptom and the symptom starts to show from the juvenile period. Recently, Avellino corneal dystrophy has been recognized since LASIK surgery becomes more popular and the cornea is damaged by UV radiation and thus the disease rapidly progression to Avellino corneal dystrophy.

After Avellino corneal dystrophy is first known in 1998 (Holland, E. J. et al., Opthalmology, 99:1564, 1992; Kennedy, S. M. et al., Br. J. Opthalmol., 80:489, 1996; Dolmetsch, A. M. et al., Can. J. Opthalmol. 31:29, 1996; Afashari, N. A. et al., Arch. Opthalmol., 119:16, 2001; Stewart, H. S., Hum. Mutat., 14:126, 1999), biochemical researches on βIG-H3 protein are recently being reported (Kim, J. E. et al., Investigative Opthalmology & Visual Science, 43:3, 2002; Park, S. J. et al., Peptides, 25:199, 2004). But until now, there has been no development of significant therapeutic agents. According to the present inventor's research, if a patient who has had LASIK surgery is identified as a heterozygote for the Avellino corneal dystrophy gene, Avellino corneal dystrophy developes or progresses rapidly (Kim, E. K. et al. Cornea, 21:223, 2002; Kim, E. K. et al., Opthalmology, 111:463, 2004).

Therefore, there is an urgent need for the development of a medicine and/or a therapeutic method which can treat Avellino corneal dystrophy, but there has not been any report, yet.

### SUMMARY OF INVENTION

Accordingly, the present inventors have made extensive efforts to develop a more effective medicine for treating Avellino corneal dystrophy, as a result, we found that hyaline granules in the cornea of a patient are effectively dissolved away when blood plasma or serum in blood is administered to corneal stromal bed of a patient.

The main object of the present invention is to provide a pharmaceutical composition for treating Avellino corneal dystrophy, which administers to the cornea of a patient with Avellino corneal dystrophy to be able to effectively remove hyaline granules.

To achieve the above object, the present invention provides a pharmaceutical composition for treating Avellino corneal dystrophy containing pharmaceutically effective amount of blood plasma or serum as an effective ingredient.

Other features and embodiments of the present invention will be more fully apparent from the following detailed description and appended claims.

### BRIEF DESCRIPTION OF DRAWINGS

FIG. 1 shows a photograph of the right eye ball of a heterozygote Avellino corneal dystrophy patient.

FIG. 2 shows photographs (magnified ×15.75) of corneal flap soaked in phosphate buffer saline (PBS) and serum for 2 days, which is obtained after LASIK surgery of a patient with Avellino corneal dystrophy.

FIG. 3 shows photographs of (A) before removing corneal flap, (B) right after removing corneal flap, and (C) after administering liquid dropping medicine containing blood plasma for 8 days, on a patient with Avellino corneal dystrophy having extremely generated white deposits due to LASIK surgery.

FIG. 4 shows photographs before removing corneal flap (left), after administering liquid dropping medicine (right), on a patient with Avellino corneal dystrophy having extremely generated white deposits due to LASIK surgery.

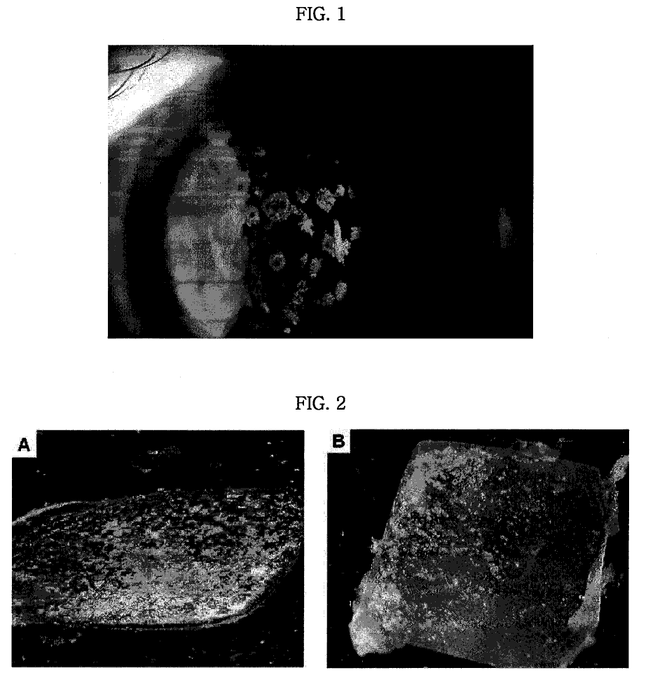

### DETAILED DESCRIPTION OF THE INVENTION AND EMBODIMENTS

The present inventors have performed extensive studies to formulate a liquid dropping medicine comprising blood plasma or serum with virus inactivation treatment and administered it to the cut section of the cornea from which corneal flap of a patient with severely developed Avellino corneal dystrophy after LASIK surgery is removed, as a result, we found that white hyaline granules in a patient's cornea dissolved away, thereby confirming that blood plasma or serum can be used as a therapeutic agent for treating Avellino corneal dystrophy.

Blood plasma typically refers to a fluid substance in mammalian blood, namely, a straw-colored liquid substance from which cells and cell fragments are separated and the substance is well known through literatures in the field (Westerman, P., Plasma Proteins, VII-1 to VII-13, 2002; Wendy, Y. C. et al., Plasma Proteins Pocket Guide, Foundation for Blood Research—the whole contents of these literatures will be cited in this application as a reference).

Blood serum used as an active ingredient in the composition of the present invention generally refers to a site where fibrinogen, clotting factors, etc. are removed from blood plasma.

Blood plasma or serum used as an active ingredient in the composition of the present invention include blood plasma or serum isolated from blood of all species of mammals including humans, for example, livestock, such as sheep, goats, pigs, horses, dogs and cattles, primates, rodents, etc.

Blood plasma or serum used in the present invention can be readily isolated from blood using conventional methods, such as centrifugation, sedimentation or filtration. Centrifugation can be carried out under suitable condition to precipitate blood cell from blood plasma. For example, centrifuging blood at about 1,400 rpm for 10 minutes is sufficient to precipitate all cell fragments containing platelets as well as red and white blood cells. Supernatant containing plasma can be easily separated from the precipitated cells by standard techniques.

Such filtration can be performed by passing blood through a filter suitable to isolate blood cells from blood plasma. The filter is preferably a microporous membrane capable of passing proteins through it easily.

Preservation methods in various forms before blood plasma or serum are used, are known, for example, fresh-frozen preparation, cryoprecipitate preparation, a lyophilized preparation or a concentrated preparation in addition to fresh liquid plasma preparation or a liquid preparation form obtained by centrifuging or sedimentating after blood was withdrawn. In the present invention, all forms of plasma or serum described above can be used.

Fresh-frozen plasma is prepared by centrifuging blood which is within 6 hours after withdrawing the blood sample at about 1,400 rpm for 15 minutes to isolate blood cells and plasma and freezing at about −40° C. to −18° C. It is preferable to use the Fresh-frozen plasma after thawing it at about 30° C.-37° C. of warm water.

Cryoprecipitated plasma is obtained by removing white precipitate (cold precipitated protein) (containing many factors, such as VIII:C, fibrinogen, XIII and fibronectin) which is generated when one unit of fresh-frozen plasma is thawed at about 4° C., and refreezing it at about −40° C. to −18° C.

For its use, cryoprecipitate preparation is thawed out by leaving it to stand in a refrigerator (1° C.-6° C.) overnight or thawed in a water bath (about 4° C. for quick use). Concentrated plasma can be used by isolating plasma from blood, concentrating after mixing the isolated plasma with a concentrating agent, such as dextranomer, SEPHDEX, dextramine, polyacrylamide, BIO-GEL P, silica gel, zeolite, DEBRISAN, crosslinked agarose, starch and alginate gel and isolating the concentrating agent from the concentrated plasma.

In one embodiment of the present invention, blood plasma or serum which can be purchased from Blood Bank can be used. For example, powdered preparations purchased from Blood Bank, liquid preparation of Invitrogen Corporation (for example, Gibco™ Chicken Serum, Gibco™ Goat Serum, Gibco™ Lamb Serum, Gibco™ Porcine Serum, Gibco™ Rabbit Serum) or serum preparation of GeminiBio-Products (USA) (for example, Chicken Serum (Cat. #100-161), Dog Serum (Cat. #100-160), Donor Donkey Serum (Cat. #100-151), Donor Goat Serum (Cat. #100-109), Donor Rat Serum (Cat. #100-155), Feline Serum (Cat. 100-153), Guinea Pig Serum (Cat. #100-130), Monkey Serum (Cat. #100-154), Mouse Serum (Cat. #100-113), Porcine Serum (Cat. #100-115), Rabbit Serum (Cat. #100-116), Rat Serum (Cat. #100-150) or Sheep Serum (Cat. #100-117) can be used. These preparations are confirmed from the test results that they are non-reactive with antibodies against hepatitis B surface antigen (HBsAg) and hepatitis C (HCV) and negative for antibodies against HIV-1 and HIV-2. All units of blood plasma used to prepare such preparations are certified free of pathogens.

When blood plasma or serum except said preparations are used, it is preferable to inactivate enveloped viruses, such as HIV, hepatitis B and HCV in blood plasma or serum to reduce the potential risk of transmission of infectious agents. The common methods among methods for inactivating blood plasma are pasteurization, dry heat treatment, vapor treatment, organic solvent/detergent mixture treatment (for example, tri(n-butyl)/phosphate/polysorbate 80), low pH (pH 4), cold ethanol fractionation, chromatography, nanofiltration. Recently UV irradiation, γ-ray irradiation, iodine treatment is being developed. It is preferable to use after blood plasma unit is subjected to continuous cycle of γ-ray irradiation, methylene blue treatment and vapor treatment to inactivate viruses which may exist in blood plasma.

As plasma or serum fractions used in the present invention, plasma or serum fractions which are powdered by heating, lyophilization or other suitable drying techniques can be used. For example, blood plasma can be used after freeze-drying at less than −40° C. for several days (e.g., about 7 days) to powderize.

Blood plasma or serum according to the present invention are liquid form or powder form, so that they can be directly applied to eyeball. In one embodiment of the present invention, the inventive pharmaceutical composition can be formulated in a liquid form by mixing blood plasma powder and physiologic saline solution at a given volume ratio and adjusting pH value to 3.5 to 8.0. This formulation is described in Remington's Pharmaceutical Science, 15th Edition, 1975, Mack Publishing Company, Easton, Pa. 18042 (Chapter 87: Blaug, Seymour) which is published prescription in all pharmaceutical chemistry.

Additionally, the present invention provides a pharmaceutical composition comprising pharmaceutically effective amount of serum together with an opthalmologically approved carrier.

Pharmaceutical composition of the present invention can contain components of adjuvants etc. including buffers, antimicrobial preserving agents, surfactants, additional pharmaceuticals, antioxidants, tonic regulators, antiseptics, thickeners and viscosity improvers.

In the present invention, any buffer among proper buffers, which harmonize with other substances of liquid preparations in the field of opthalmology and doesn't show harmful characteristic or toxicity that can damage eyes, can be used as the buffer. The proper buffers include boric acid, sodium boric acid, sodium phosphate (including 1, 2 and 3 basic phosphate, such as 1 basic sodium phosphate 1 hydrate, 2 basic sodium phosphate 7 hydrate and mixtures thereof). Any other proper buffers can be used to stabilized pH level of the ophthalmic liquid medicine by conferring physiological pH approved for ophthalmic liquid medicines. Since said buffers are just examples and these buffers are well known in opthalmologic field, a person skilled in the art can choose proper buffers that can be used for the composition of the present invention.

In the present invention, the preferable examples of the antimicrobial preserving agent include benzalcholnium chloride, timerosal, chlorobutanol, methyl paraben, prophyl paraben, phenylethyl alcohol, edetate disodium, sorbic acid and ONAMER M.

Especially, it is preferable to use benzalcholnium chloride as a antimicrobial preserving agent in a pharmaceutical composition of the present invention, because benzalcholnium chloride increases the possibility of dissolving hyaline in the cornea by functioning to weaken the boundary of the cornea epithelial cells to facilitate cornea permeation of serum in addition to functioning as a preserving agent. It is preferable to use the above antimicrobial preserving agents at an amount of about 0.001% by weight-1.0% by weight based on the total weight of composition. Especially, it is preferable to add at an amount of about 0.01% by weight-0.10% by weight when benzalcholnium chloride is used.

The pharmaceutical composition of the present invention can be prepared in various formulations, such as liquid, suspension, emulsion, gel and a solid form of insert into eyes as a person skilled in the art can easily recognizes.

pH of the pharmaceutical composition of the present invention is preferable to be pH 6.8-8.0 which corresponds to pH of eye liquid or at which eyes have resistance without uncomfortableness or inflammation, and more preferably pH 7.0-7.8. To stabilize an ophthalmic liquid medicine at a desirable pH level, small amount of effective buffer is mixed. An effective amount of buffer administered to buffer an ophthalmic liquid medicine at about pH 6.8-8.0 can be broadly varied and determined according to a specific buffer used and a chemical composition of the pharmaceutical composition. But, to stabilize this liquid medicine at approved physiological pH, preferable result can be obtained when the amount of buffer mixed in the ophthalmic liquid medicine is about 0.05-1% weight/volume.

The osmotic pressure of the pharmaceutical composition of the present invention is preferable generally about 1-400 mOsM, and more preferably 260-340 mOsM. If necessary, the osmotic pressure can be adjusted using salt or drug vehicle approved in physiology and opthalmology. NaCl is suitable to approach physiological saline solution. The amount of NaCl added is preferably 0.01-1% by weight based on the total weight of the composition, it is more preferable to be added in a range of 0.05%-0.45% by weight. To achieve the osmotic concentration of the above range, an equivalent amount of at least one salt comprised of anions, such as potassium, ammonium and cations, such as chloride, citrate, ascorbate, borate, phosphate, bicarbornate, sulfate, tiosulfate, bisulfate, sodium bisulfate, ammonium sulfate can be used together with NaCl or in place of NaCl. Also, sugar, such as mannitol, denstrose, sorbitol, glucose can be used to adjust the osmotic concentration.

In the present invention, water which is used for diluting blood plasma or serum must be the water without any physiologically or opthalmologically harmful substance. Typically, purified water or desalinized water is used.

It is preferable to directly administer the inventive pharmaceutical composition to the affected part of patient's body at an interval of one or two hours 16-8 times per day (except during the sleeping hours), and the preferable amount of administration is 20-2001, and more preferably 40-100 μl.

In one aspect, the effective amount of the freeze-dried blood plasma or serum used in pharmaceutical composition of the present invention can be used at about 0.001-90% based on weight, more preferably 0.005-50%.

In another aspect of the present invention, when commercially available blood plasma or serum is used in the inventive pharmaceutical composition, 1-95% by weight can be used based on weight.

### EXAMPLES

The present invention will hereinafter be described in further detail by examples. It will however be obvious to a person skilled in the art that these examples are given for illustrative purpose only, and the scope of the present invention is not limited to or by the examples.

Also, according to below examples and experimental examples, although the corneal flap of a patient with Avellino corneal dystrophy is removed to administer a pharmaceutical composition comprising blood plasma or serum to the cut section of the cornea, it is obvious to a person skilled in the art that it can be effective for a patient without surgery of removing corneal flap from the fact showing continuous dissolution effect of hyaline granules even after 3 days when the cut section is covered with epithelium.

### Example 1

### Isolation of Liquid Blood Plasma from Whole Blood

After fresh-frozen blood (Central Blood Center) derived from a person determined negative from the detection result for potential pathogens including HIV, HCV and hepatitis B, was thawed out at 30° C. in a water bath and centrifuged at 3,000 rpm, for 10 minutes to isolate supernatant, straw color blood plasma except precipitates (red blood cell, white blood cell, etc.).

### Example 2

### Isolation of Liquid Serum from Whole Blood

Fresh-frozen blood derived from a person determined negative from the detection result for potential pathogens including HIV, HCV and hepatitis B was collected in glass tube without adding anticoagulant (for example, EDTA, heparine). The blood was left to stand at 4° C. overnight, and removed the formed clot using Pasteur pipette. Blood from which the clot is removed was centrifuged at 4,000 rpm, 4° C. for 20 minutes to isolate supernatant blood serum.

### Example 3

### Virus Inactivation in Blood Plasma and Serum

Virus which can exist in the blood plasma or serum was inactivated by performing the following three methods continuously.

### (a) γ-Ray Irradiation

Liquid blood plasma or serum was irradiated with total 25 kGy of γ-ray at the intensity of 1.8 kGy/hr using ⁶⁰Co at 15° C.

### (b) Methylene Blue Treatment

The liquid blood plasma or serum irradiated by the γ-ray was added with Methylene blue to the final concentration of 1 μM and irradiated by white light for 1 hr at 60,000 lux. Residual methylene blue was filtered and removed and the mixture was frozen for 8 hrs at −80° C. and dried for 7 days at −48° C. to freeze-dry.

### (c) Vapor Treatment

Freeze-dried blood plasma or serum was filtered by sieve, and ground to be homogenized, followed by slowly injecting vapor to 8% (w/w) of water content in stainless steel tank. After the blood plasma treated with vapor was transferred to stainless steel cylinder charged with dry nitrogen to remove oxygen, it was heated for 10 hrs, at 60° C.

### Example 4

### Production of the Liquid Dropping Medicine for Treating Avellino Corneal Dystrophy

After the blood plasma or serum in which viruses are inactivated in example 3 was mixed with physiological saline solution in a volume ratio of 10:1, proper amount of mixed solution was added to dropping medicine showed in table 1. pH of the liquid dropping medicine was adjusted to pH 7.4 using 1 N HCl or IN NaOH. A residual liquid dropping medicine was deep-frozen in freeze-drying bottle, vial, container, tray or other storage container.

TABLE 1
The component of the liquid dropping medicine for treating
Avellino corneal dystrophy comprising blood plasma or serum
as an effective ingredient.

| Component | Weight percent |
|---|---|
| Blood plasma sol. or Serum sol. (10%) | 50 |
| Benzalconium chloride (BAK)(50%) | 0.02 |
| Sodium borate | 0.035 |
| NaCl | 0.096 |
| KCl | 0.097 |
| EDTA | 0.030 |
| Purified water | Proper amount until 100% |

### Experimental Example 1

### The Effect of Dropping Medicine for Treating Avellino Corneal Dystrophy on Removing Granular Corneal Opacities

A peripheral blood was withdrawn from a patient having white granular corneal opacities between corneal flap and stromal bed after LASIK to perform DNA analysis as described in publication (Wan, X. H et al., Cornea, 21:223-6, 2002). As a result, the patient was identified as a heterozygote for the Avellino corneal dystrophy gene having R124H mutation by exon 4 sequence analysis of TGFBI gene.

The corneal flap obtained from the patient with Avellino corneal dystrophy was soaked in phosphate buffer saline and 1:10 dilution of serum of the Example 4 for 2 days. In FIG. 2, A shows the photograph of the corneal flap in phosphate buffer solution and B shows the photograph of the corneal flap in 1:10 dilution of serum of the Example 4. As shown in FIG. 2, it was confirmed that white hyaline opacities existed in the corneal flap in phosphate buffer solution, whereas significant amount of white hyaline opacities were dissolved away in serum solution diluted 1:10.

### Experimental Example 2

### Therapeutic Effect of the Liquid Dropping Medicine for Treating Avellino Corneal Dystrophy

A peripheral blood was withdrawn from a patient having white granular corneal opacities between corneal flap and stromal bed after LASIK surgery, as described in publication (Wan, X. H et al., Cornea, 21:223-6, 2002), to perform DNA analysis. As a result, the patient was identified as a heterozygote for the Avellino corneal dystrophy gene having R124H mutation by exon 4 sequence analysis of TGFBI gene.

After removing the corneal flap from a patient's eye to expose posterior remaining stromal surface, 1 drop (about 50 μl) or 2 drops of liquid dropping medicine comprising blood plasma or serum prepared in example 4 was dropped one time at an interval of 1 hour for 20 days except sleeping time.

It took 3 days for the corneal epithelium to grow and cover the exposed posterior remaining stromal surface. The dropping was continued for an additional 17 days after the posterior remaining stromal surface was covered with epithelium, which leads to the confirmation that the effect lasts.

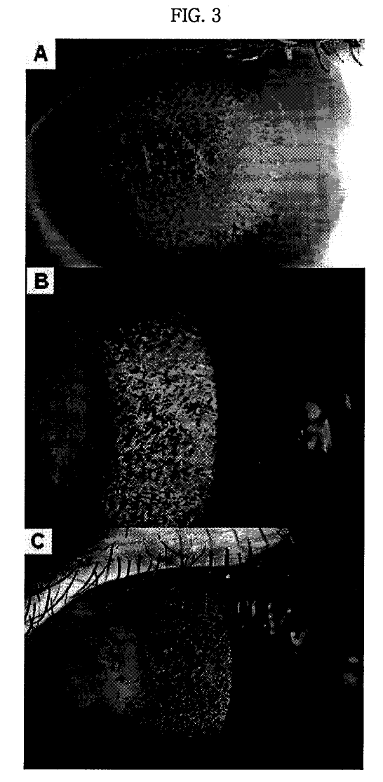

FIG. 3 shows the photographs of (A) before removing the corneal flap, (B) right after removing the corneal flap, and (C) after administering liquid dropping medicine containing blood plasma for 8 days, on a patient with Avellino corneal dystrophy having extremely increased white granule corneal opacities due to LASIK surgery. As shown in FIG. 3, it was suggested that when the corneal flap of the patient with a lot amount of white granule corneal opacities in the cornea after LASIK surgery was removed and the liquid dropping medicine containing blood plasma was administered, the granule corneal opacities decreased.

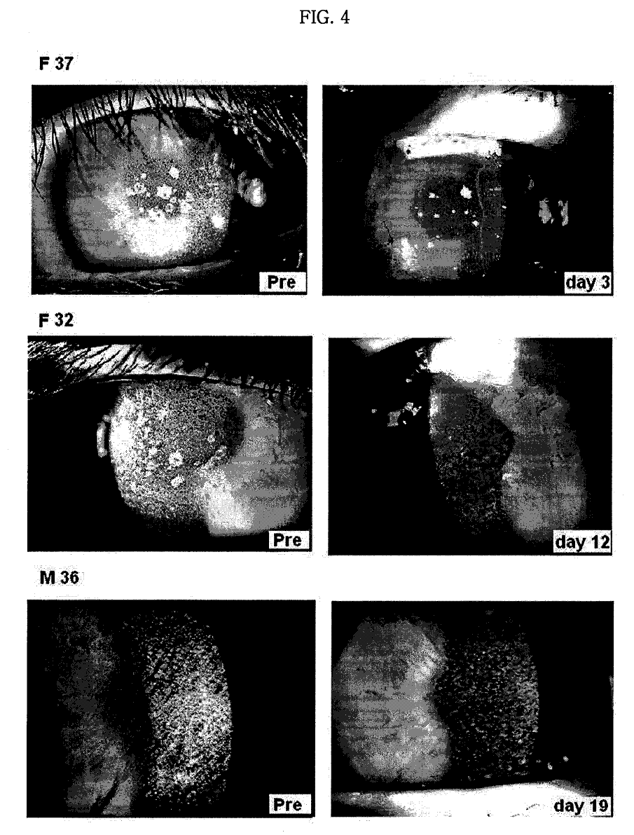

FIG. 4 shows the photographs before removing the corneal flap (left), after administering liquid dropping medicine containing serum (right), on a patient with Avellino corneal dystrophy having extremely increased granule corneal opacities after LASIK surgery. The first column of FIG. 4 shows the photographs of the left eye of a 37 year old female patient, in which the right side is a photograph of one eye before removing the corneal flap and the left side is a photograph of one eye after administering liquid dropping medicine containing blood serum for 3 days after removing the corneal flap. The second column of FIG. 4 shows the photographs of the right eye of a 32 year old female patient, in which the right side is a photograph before removing the corneal flap and the left side is a photograph after administering liquid dropping medicine containing blood serum for 12 days after removing the corneal flap. The third column of FIG. 4 shows the photographs of the left eye of a 36 year old male patient, in which the right side is a photograph before removing the corneal flap and the left side is a photograph administered with liquid dropping medicine containing blood serum for 19 days after removing the corneal flap. As shown in FIG. 4, it was suggested that when the corneal flap of the patient having lots of white granule corneal opacities in the cornea after LASIK surgery was removed to administer the liquid dropping medicine containing blood serum, granular corneal opacities remarkably decreased.

Although the present invention has been described in detail with reference to the specific features, it will be apparent to those skilled in the art that this description is solely for a preferred embodiment and does not limit the scope of the present invention. Thus, the substantial scope of the present invention will be defined by the appended claims and equivalents thereof.

### INDUSTRIAL APPLICABILITY

As described above in detail, the present invention has an effect of providing a pharmaceutical composition for treating Avellino corneal dystrophy, which is administered to the cornea of the patient with Avellino corneal dystrophy to be able to remove hyaline granules effectively. The pharmaceutical composition of the present invention has an effect of improving symptoms by dissolving away hyaline granules in the cornea of the patient with severe Avellino corneal dystrophy due to LASIK surgery.

1. A pharmaceutical composition for treating Avellino corneal dystrophy, comprising a pharmaceutically effective amount of blood plasma or serum as an effective ingredient.

2. The pharmaceutical composition according to claim 1, which additionally comprises one or more adjuvant selected from the group consisting of buffers, anti-microbial preserving agents, surfactants, antioxidants, tonic regulators, antiseptics, thickeners and viscosity improvers.

3. The pharmaceutical composition according to claim 2, wherein the antimicrobial preserving agent is selected from the group consisting of benzalcholnium chloride, timerosal, chlorobutanol, methyl paraben, prophyl paraben, phenylethyl alcohol, edetate disodium, sorbic acid and ONAMER M.

4. The pharmaceutical composition according to claim 3, wherein said antimicrobial preserving agent is benzalconium chloride.

5. The pharmaceutical composition according to claim 1, wherein said blood plasma or serum is treated with at least one virus inactivation selected from the group consisting of γ-ray irradiation, methylene blue treatment and vapor treatment.

6. The pharmaceutical composition according to claim 1, wherein said blood plasma or serum is freeze-dried.

7. The pharmaceutical composition according to claim 6, containing the freeze-dried blood plasma or serum at a concentration of from 0.005 to 50% by weight, based on weight of the pharmaceutical composition.

8. The pharmaceutical composition according to claim 1, in a liquid, suspension, emulsion, gel or powder form.
